(19) United States
(12) Patent Application Publication
(10) Pub. No.: US 2003/0203060 A1
(43) Pub. Date: Oct. 30, 2003

(54) Topical medicine and process and apparatus for making same

(76) Inventor: Joe Ajrulahi, Garden City Beach, SC (US)

Correspondence Address: B. Craig Killough, Barnwell Whaley Patterson & Helms, LLC, P.O. Drawer H, Charleston, SC, 29402-0197, US

(21) Appl. No.: 10/449,427

(22) Filed: May 30, 2003

Related U.S. Application Data
(63) Continuation-in-part of application No. 09/905,770, filed on Jul. 13, 2001, now abandoned.

(51) Int. Cl. A61K 35/78

(52) U.S. Cl. 424/760

(57) ABSTRACT

A medicine that is topically applied comprises vegetable or fruit matter, including materials resulting from extracts of fruit or vegetable juices, and including juices have undergone a fermentation process. These ingredients are combined and processed by a device that applies a current through a metallic electrode, which contributes metal to the medicine. In another process, the device may be used to remove alloys from metals. Alloys may be removed from gold, such as gold jewelry, and from other metals such as copper.

### FIELD OF THE INVENTION

This invention relates to a topical medicine preparation. A process for making the topical medicine preparation, and an apparatus for practicing the process are also presented.

### SUMMARY OF THE INVENTION

The present invention is a medicine that may be topically applied. The medicine may comprise other vegetables, and fruit and/or vegetable juices, including fruit or vegetable juices that have undergone a fermentation process. These ingredients are combined and processed using electrolysis according to the invention, wherein an electrode contributes very fine metallic particles to the juice. In another process, the device disclosed herein may be used to remove alloys from metals. Alloys may be removed from gold, such as gold jewelry, and from other metals such as copper.

### DESCRIPTION OF THE DRAWINGS

FIG. 1 is a schematic diagram of an embodiment of an apparatus that may be used to prepare the topical medicine according to the method of the present invention, with juice from a plant present therein.

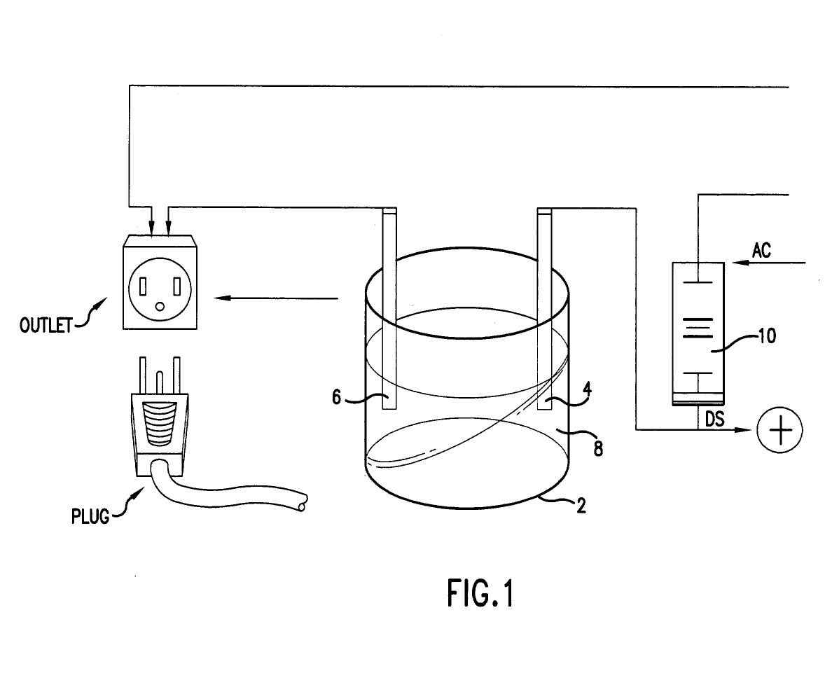

FIG. 2 is a schematic diagram of an embodiment of an apparatus that may be used to prepare the topical medicine according to the method of the present invention, with the powder from which the topical medicine is prepared present below a supernatant liquid.

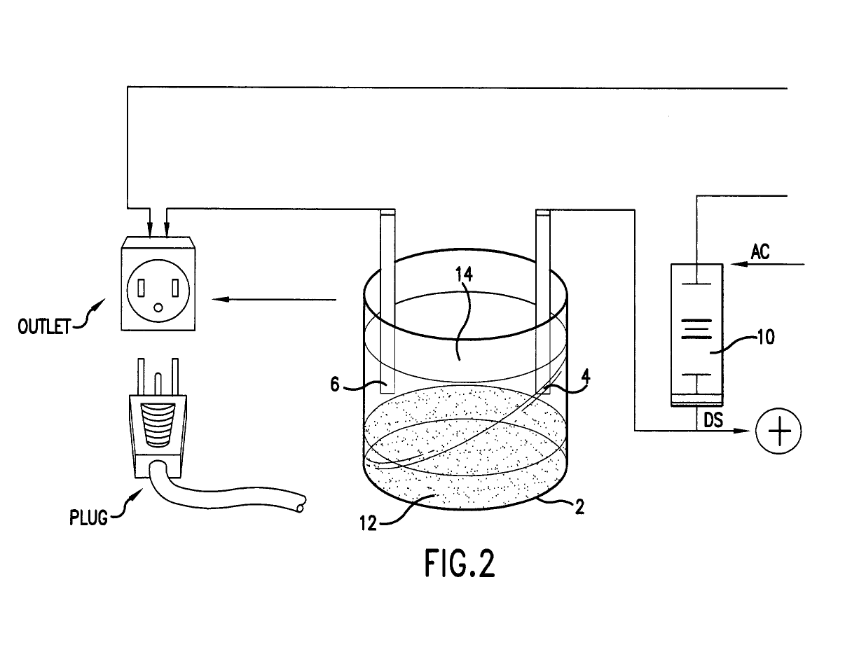

FIG. 3 is a schematic diagram of an embodiment of an apparatus that may be used to prepare the topical medicine according to the method of the present invention.

### DETAILED DESCRIPTION OF THE PREFERRED EMBODIMENT

In a preferred embodiment, jalapeno pepper juice is obtained. The jalapeno pepper juice may be obtained by extracting the juice from jalapeno peppers by known means. For example, a food processor or similar device may be used to grind the jalapeno pepper, with the ground jalapeno pepper subsequently squeezed to extract the juice. The juice is then strained to obtain the juice.

The jalapeno pepper juice is then placed into a beaker 2. The beaker is preferred to be formed of a non-conductive material, such as glass. A metallic electrode is inserted into the beaker. In the preferred embodiment, the electrode 4 is copper. A second, electrically conductive electrode 6 is also placed into the beaker. The second electrode may comprise copper. The electrodes are positioned to contact the jalapeno pepper juice 8, but do not contact each other.

The electrodes are connected to a current source, such as a direct current source. The direct current may be transformed from standard household current, which, in the United States, is 110 volts alternating current by means of a transformer. Two hundred (200) watts of AC power may be provided to the transformer. Higher, or lower, voltages and wattages may be used. As shown in the drawing figures, a semi-conductor 10 may be used as a half wave rectifier to which household current is used to supply a current to the electrode 4. A silicon controlled rectifier could be used.

The current is provided to the jalapeno pepper juice though electrode 4. Current is supplied for the desired period of time. Typically, current is supplied until the desired quantity of the medicinal powder is present in the bottom of the beaker. The powder comprises copper, which is produced from the electrode, and the jalapeno pepper product. It should be understood that the resulting powder comprises both and copper and non-liquid jalapeno pepper, and that the components are not separate and distinguishable to the naked eye. The process as described produces a powder 12 comprising approximately 50% copper and 50% jalapeno pepper, when approximately ⅓ to ½ of the content in the beaker is powder and the remainder of the content is a supernatant liquid having a bluish green tint.

By empirical observation, four ounces of powder can be obtained by applying 200 watts of power for approximately one hour. An increase in power will reduce the time required to produce the same weight of powder

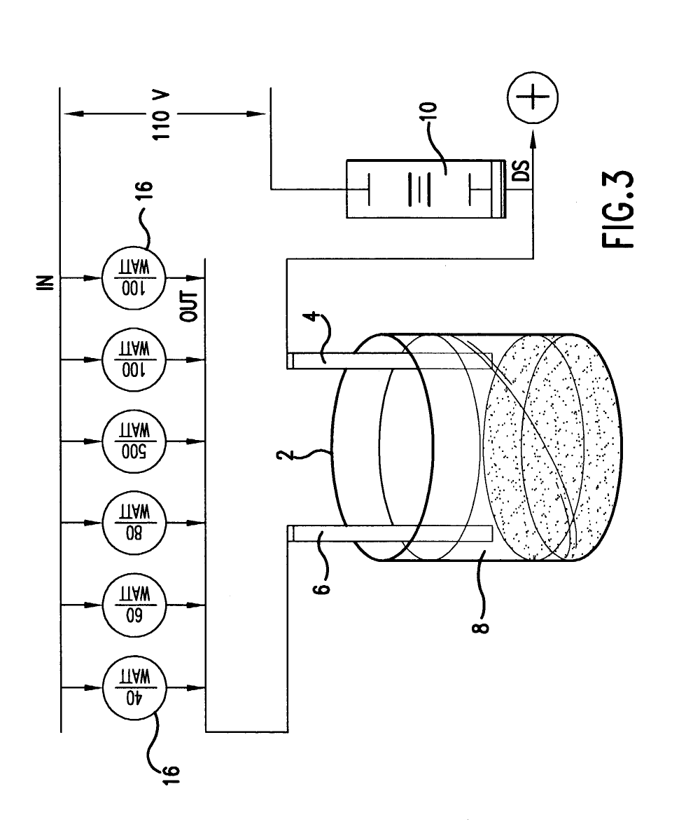

Referring to FIG. 3, a set of light bulbs 16 are in a parallel circuit. The light bulbs are preferred to be of varying wattages, as shown. The light bulbs are screwed in, or unscrewed, as desired, and are thereby used to vary the voltage across the electrodes as desired. In practice, light bulbs are introduced until the light bulbs glow with slightly less brightness than if they were in a light fixture, rather than in the present circuit. When this level of brightness is achieved, the voltage across the electrodes is deemed to be correct for the volume of juice to be processed. A rheostat or voltage divider could be used in place of the light bulbs.

The electrode that is connected to the rectifier is copper and contributes copper to the production of the powder. The second electrode conducts electricity. It may, but need not, comprise copper.

The resulting powder composition is topically applied as a medicine. It may be combined with an inert vehicle for topical application. The vehicle may be a cream, emollient, lotion or salve, such as petroleum jelly.

Other fruit or vegetable products may be used alone or in combination, although jalapeno pepper juice has been demonstrated to achieve particularly good results.

The electrolytic process described herein produces a composition having very small particles of metal, with the metal particles being less than 1 micron. For example, when a copper electrode is used, the resulting particles of the deposited copper are shown by an X-ray diffraction test to be less than 0.00004 inch. As the particle sizes of metals become very fine, the properties of the metal change. In particular, as the surface ratio becomes larger, the metal becomes more reactive. When used in medicine, the fine particle size allows the metal to be absorbed rapidly and efficiently into the skin and the system of the user. In the present invention, a very fine colloidal suspension of metal is produced by electrolysis, which forces the oxidation of the metal, and supersaturates the electrolyte solution with copper. The copper then precipitates out of the solution as a very fine copper suspension.

During the first stage of the electro generative process of the invention, the metal from the electrode is transferred by oxidation due to the flow of electrons into solution as a free metal, such as copper ion. “Oxidation” (defined as causing an increase in valence) during this stage produces the free ion, and does not mean that a copper oxide is formed. In this stage, there is essentially no copper suspension or powder in the liquid. This liquid could be referred to as an “intermediate liquid”. The second stage occurs after the solution becomes supersaturated with metal. During this stage, the metal starts to form a very fine powder (less than 1 micron size) that is in suspension.

There may also be compounds formed as oxides, such as copper oxide, or as other compounds during both stages. The fruit or vegetable juice with the metal, such as copper, ions produced during the first stage act as a dispersant for stage 2. In actuality, the two stages may overlap slightly, due to the lack of perfect mixing.

Thus, there are two forms of metal, such as copper, in the medicine. The first form is metal ions in solution. This solution then acts as a dispersant or “dispersion phase”. The second form is a very fine metal powder in suspension. The powder acts as a “dispersed phase”.

As the size of a chemical becomes smaller in diameter it becomes more reactive to the square. This is due to the increased surface area. The smaller size or increased surface area can significantly accelerate a chemical reaction, and even the properties of chemicals can even change. Small size fractions are able to penetrate into places that larger sizes are not be able to go. In the case of the medicine produced by this process, the small metal particles in ion or very fine powder form can penetrate deeply through the skin and into places that larger particles cannot enter.

A problem is placement of the metal in the right place and with a fine enough size to cause a rapid impact. Oral medications or injections tend to spread out throughout the entire body with little getting to the proper location. The application of a sufficient quantity of metal to the area needed, by oral or injection methods, could be toxic and cause a patient to become sick or even die. In order to apply the medicine locally, it needs to be able to penetrate the skin and flesh and concentrate itself locally. The very fine size metal particles of the product of the present process presents an appropriate vehicle to accomplish this goal.

The surface ratio of the copper produced by this process is about 0.03 square meters per gram or larger. In this medicine, the size of the metal not only approaches the characteristics of a catalyst but it is also the reactant. At this surface ratio the reactions of the metal as a medicine are unique.

The colloidal suspension of metal is produced in the presence of fruit or vegetable juice. The capsaicin that is present in jalapeno peppers and other peppers provides warming of the skin, and the underlying muscles and circulatory system, and provides relief for aches and pains. The extremely small metallic particles, such as those in copper, are absorbed through the skin, and the efficacy of the copper is improved by the warming sensation provided by the capsaicin. The combination of capsaicin and the extremely small particles produced by the electrolytic process, when provided by a cream or emollient carrier, yields an effective topically applied medicine. The medicament may also be used for treatment of dermatological conditions, including those related to abnormal pigmentation.

The device depicted in the drawing figures may be used to refine precious metals. An alloyed electrode comprising a precious metal, such as gold alloy, may be introduced to a charge from the transformer or rectifier. The electrode is present in a beaker comprising salt water, and opposite another electrode as shown in the drawing figures. Current is applied. After the electrode is deposited into the salt water by the current, the current is terminated, and nitric acid is introduced into the beaker. Water is then added. The alloy is poured off with the water, and pure gold remains in the beaker. The process as described will separate the gold from the alloy. Other precious metals may be similarly refined.

What is claimed is:

1. A process of producing a medicine for topical application comprising metal, said process comprising the steps of:
a) obtaining juice from a plant;
b) placing said juice in a non-electrically conductive container;
c) introducing a first electrically conductive and metallic electrode and a second electrically conductive electrode into said juice in said container;
d) applying a current that is not produced by said first electrically conductive and metallic electrode and said second electrode to said second electrode and said first electrically conductive and metallic electrode, wherein particles of metal from said first electrically conductive and metallic electrode are deposited into said juice in said container;
e) continuing to apply said current until a desired amount of material is produced that comprises components from said juice and from said first electrically conductive and metallic electrode; and
f) producing a medicine from said material.

2. A process of producing a medicine comprising metal, said process comprising the steps of:
a) obtaining juice from a plant;
b) placing said juice in a non-electrically conductive container;
c) introducing a first electrically conductive and metallic electrode and a second electrically conductive electrode into said juice in said container;
d) applying a negatively charged direct current to said second electrode and a positively charged direct current to said first electrically conductive and metallic electrode, wherein particles of metal from said first electrically conductive and metallic electrode are deposited into said juice in said container;
e) continuing to apply said current until a desired amount of material is produced that comprises components from said juice and said first electrically conductive and metallic electrode; and
f) producing a medicine from said material.

3. A process of producing a medicine comprising metal as described in claim 1, wherein said first electrically conductive and metallic electrode comprises copper.

4. A process of producing a medicine comprising metal as described in claim 2, wherein said first electrically conductive and metallic electrode comprises copper.

5. A process of producing a medicine comprising metal as described in claim 1, wherein said juice comprises capsaicin.

6. A process of producing a medicine comprising metal as described in claim 2, wherein said juice comprises capsaicin.

7. A process of producing a medicine comprising metal as described in claim 5, wherein said juice is obtained from jalapeno pepper.

8. A process of producing a medicine comprising metal as described in claim 6, wherein said juice is obtained from jalapeno pepper.

9. A process of producing a medicine comprising metal as described in claim 1, wherein said current is applied until after said juice becomes supersaturated with particles of metal.

10. A process of producing a medicine comprising metal as described in claim 1, wherein said current is applied until after said juice becomes supersaturated with particles of metal.

11. A process of producing a medicine comprising metal as described in claim 1, wherein said medicine is produced by combining said material with an inert vehicle for topical application.

12. A process of producing a medicine comprising metal as described in claim 2, wherein said medicine is produced by combining said material with an inert vehicle for topical application.

13. A process of producing a medicine comprising metal as described in claim 1, wherein said first electrically conductive and metallic electrode is corrosive in the process described in claim 1.

14. A process of producing a medicine comprising metal as described in claim 1, wherein said first electrically conductive and metallic electrode is corrosive in the process described in claim 2.

15. A medicine comprising metal produced by the process of claim 1.

16. A medicine comprising metal produced by the process of claim 2.

17. A medicine comprising metal produced by the process of claim 3.

18. A medicine comprising metal produced by the process of claim 4.

19. A medicine comprising metal produced by the process of claim 5.

20. A medicine comprising metal produced by the process of claim 6.

21. A medicine comprising metal produced by the process of claim 7.

22. A medicine comprising metal produced by the process of claim 8.

23. A medicine comprising metal produced by the process of claim 9.

24. A medicine comprising metal produced by the process of claim 10.

25. A medicine comprising metal produced by the process of claim 11.

26. A medicine comprising metal produced by the process of claim 12.

27. A medicine comprising metal produced by the process of claim 13.

28. A medicine comprising metal produced by the process of claim 14.
